(19) United States
(12) Patent Application Publication
(10) Pub. No.: US 2025/0078993 A1
(43) Pub. Date: Mar. 6, 2025

(54) DEVICE MANAGEMENT SYSTEM

(71) Applicant: Surgio Health, Scottsdale, AZ (US)

(72) Inventors: Jeff Claeson, Cave Creek, AZ (US); Anthony Passalacqua, Peoria, AZ (US); Jeff Wertz, Scottsdale, AZ (US)

(73) Assignee: Surgio Health, Scottsdale, AZ (US)

(21) Appl. No.: 18/458,539

(22) Filed: Aug. 30, 2023

(51) Int. Cl. G16H 40/40; G16H 40/20

(52) U.S. Cl. CPC G16H 40/40; G16H 40/20

(57) ABSTRACT

Disclosed are systems, apparatuses, methods, and computer readable medium for managing devices within a medical, scientific, academic, or other facility. A method includes: requesting a set of medical devices from an external service using a service; receiving consignment information of the set of medical devices from the service, wherein the external service provides information to the medical instrument service to trigger the consignment information; when the set of medical devices is received, obtaining identification information of the set of medical devices and providing the identification information and status information to a repository; receiving a label for coupling to the label to the set of medical devices, the label comprising medical device information and a barcode with identifier information, obtaining information encoded into the barcode; and transmitting, to the medical instrument service, operation information associated with a function of the set medical devices.

### TECHNICAL FIELD

The present disclosure generally relates to a device management system. In some examples, aspects of the present disclosure are related to a device management system for managing medical, scientific, and other equipment and various operations such as testing and maintenance.

### BACKGROUND

Medical facility management involves the effective coordination and organization of resources, personnel, and operations to ensure the smooth functioning of a healthcare facility. This can involve overseeing various departments such as administration, patient care, facility maintenance, and finance. The primary goal of medical facility management is to ensure that patients receive high-quality care in a safe and comfortable environment. In addition to patient care, medical facility management also involves ensuring that the facility meets all legal and regulatory requirements, such as maintaining proper documentation and complying with healthcare laws and regulations.

Medical device management involves the safe and effective handling and maintenance of medical equipment and devices used in healthcare facilities. This can include everything from diagnostic equipment like X-ray machines and ultrasound scanners to surgical tools and monitoring devices. Proper management of medical devices involves regular maintenance and calibration, as well as ensuring that they are properly sterilized and stored. In addition, medical device management also involves keeping accurate records of equipment use and maintenance to ensure compliance with regulatory requirements. However, in many cases, the medical facility relies on third-party services to provide equipment due to various limitations of the medical facility.

### SUMMARY

In some examples, systems, and techniques are described for managing devices across scientific, technical, medical, and other facilities. The systems and techniques can improve operations, identify operations deficiencies, and improve asset utilization using machine-based techniques that cannot feasibly be performed manually.

Disclosed are systems, apparatuses, methods, computer readable medium, and circuits for managing devices, equipment, and instruments. According to at least one example, a method includes: requesting a set of medical devices from an external service using a medical instrument service; receiving consignment information of the set of medical devices from the medical instrument service, wherein the external service provides information to the medical instrument service to trigger the consignment information; when the set of medical devices is received, obtaining identification information of the set of medical devices and providing the identification information and status information to a repository; receiving a label for coupling to the label to the set of medical devices, the label comprising medical device information and a barcode with identifier information, wherein the medical device information comprises identification of medical events and at least one person associated with the event, and wherein a status of the set of medical devices is changed in the medical instrument service in response to providing the label to the computing device; obtaining information encoded into the barcode; and when the identifier information from the barcode is obtained, transmitting operation information to the medical instrument service associated with a function of the set medical devices, wherein the medical instrument service updates the status of the set of medical devices, and wherein the medical instrument service updates the status of the set of medical devices based on information included in the operation information; performing various operations within the facility in conjunction with a device management service; and returning the set of medical devices to the medical instrument service. For example, the device management system requests a set of medical devices from an external service using a medical instrument service; receives consignment information of the set of medical devices from the medical instrument service, wherein the external service provides information to the medical instrument service to trigger the consignment information; when the set of medical devices is received, obtains identification information of the set of medical devices and providing the identification information and status information to a repository; receives a label for coupling to the label to the set of medical devices, the label comprising medical device information and a barcode with identifier information, wherein the medical device information comprises identification of medical events and at least one person associated with the event, and wherein a status of the set of medical devices is changed in the medical instrument service in response to providing the label to the computing device; obtains information encoded into the barcode; and when the identifier information from the barcode is obtained, transmits operation information to the medical instrument service associated with a function of the set medical devices, wherein the medical instrument service updates the status of the set of medical devices, and wherein the medical instrument service updates the status of the set of medical devices based on information included in the operation information; performs various operations within the facility in conjunction with a device management service; and returns the set of medical devices to the medical instrument service.

In another example, a device management system for managing devices, equipment, and instruments is provided that includes a storage (e.g., a memory configured to store data, such as virtual content data, one or more images, etc.) and one or more processors (e.g., implemented in circuitry) coupled to the memory and configured to execute instructions and, in conjunction with various components (e.g., a network interface, a display, an output device, etc.), cause the device management system to: request a set of medical devices from an external service using a medical instrument service; receive consignment information of the set of medical devices from the medical instrument service, wherein the external service provides information to the medical instrument service to trigger the consignment information; when the set of medical devices is received, obtain identification information of the set of medical devices and providing the identification information and status information to a repository; receive a label for coupling to the label to the set of medical devices, the label comprising medical device information and a barcode with identifier information, wherein the medical device information comprises identification of medical events and at least one person associated with the event, and wherein a status of the set of medical devices is changed in the medical instrument service in response to providing the label to the computing device; obtain information encoded into the barcode; and when the identifier information from the barcode is obtained, transmit operation information to the medical instrument service associated with a function of the set medical devices, wherein the medical instrument service updates the status of the set of medical devices, and wherein the medical instrument service updates the status of the set of medical devices based on information included in the operation information; perform various operations within the facility in conjunction with a device management service; and return the set of medical devices to the medical instrument service. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

Further disclosed herein is a non-transitory computer readable medium storing instructions that when executed by at least one processor, cause the at least one processor to perform operations for device management. The operations may include transmitting a request for a set of medical devices from an external service using a medical instrument service and receiving consignment information of the set of medical devices from the medical instrument service, wherein the external service provides information to the medical instrument service to trigger the consignment information. When the set of medical devices is received, the operations may further include, receiving identification and status information of the set of medical devices in a repository. The operations may further include providing a label to couple to the set of medical devices, the label comprising medical device information and a barcode with identifier information, wherein the medical device information comprises identification of medical events and at least one person associated with the event, and wherein a status of the set of medical devices is changed in the medical instrument service in response to providing the label to the at least one processor. The operations may further include obtaining information encoded into the barcode, and in response to obtaining the identifier information from the barcode, transmitting, to the medical instrument service, operation information associated with a function of the set of medical devices, wherein the medical instrument service updates the status of the set of medical devices wherein the medical instrument service updates the set of medical devices based on information included in the operation information.

In some aspects, the operations may further include transmitting a query requesting identification of a first medical device within a facility associated with the request for the set of medical devices, and receiving identification of a second set of medical devices within the facility including the first medical device. In another aspect, the operations may include transmitting service information to the medical instrument service related to servicing the set of medical devices, wherein the servicing information is provided to the external service to trigger at least one operation for at least one medical device of the set of medical devices. In an aspect, the operations may further include requesting status information from the medical instrument service pertaining to at least one status associated with sets of medical devices within a facility associated with the request for the set of medical devices, and receiving the status information identifying performance of the at least one status within the facility. In an aspect, the status information includes performance associated with at least one of at least one of sterilization operations, consignment operations, maintenance operations, or internal operations within the facility.

In various aspects, the operations may further include displaying information related to a current operation associated with the set of medical devices in response to obtaining the identifier information from the barcode. In another aspect, the operations may further include transmitting a request for an analysis of an operation type from the medical instrument service, and receiving previous information related to the operation type from the medical information, wherein the previous information includes identification of a configuration of each operation type, and wherein the configuration identifies at least one set of medical devices. In an aspect, the analysis of the operation type may include a parameter associated with at least one of a patient, a medical condition, and an operation type. In another aspect, the previous information may include at least one medical operator.

The foregoing, together with other features and aspects, will become more apparent upon referring to the following specification, claims, and accompanying drawings.

### BRIEF DESCRIPTION OF THE DRAWINGS

In order to describe the manner in which the various advantages and features of the disclosure can be obtained, a more particular description of the principles described herein will be rendered by reference to specific embodiments thereof which are illustrated in the appended drawings. Understanding that these drawings depict only example embodiments of the disclosure and are not to be considered to limit its scope, the principles herein are described and explained with additional specificity and detail through the use of the drawings in which:

FIG. 1 is a conceptual diagram illustrating a device management system, in accordance with some aspects of the disclosure;

FIG. 2 is a conceptual flow diagram illustrating an example of operations performed by a third-party service associated with a device management system in accordance with some aspects of the disclosure;

FIG. 3 is a conceptual flow diagram illustrating an example of operations performed by a facility associated with a device management system in accordance with some aspects of the disclosure;

FIG. 4 is a functional block diagram of a device management system in accordance with some aspects of the disclosure;

FIG. 5 illustrates an example label that is provided by a device management system in accordance with some aspects of the disclosure;

FIG. 6 illustrates various timelines that describe the operation of a device management system in connection with a medical facility in accordance with some aspects of the disclosure;

FIG. 7 illustrates an example method of a device management system in accordance with some aspects of the disclosure;

FIG. 8 is a conceptual diagram illustrating an architecture and deployment of a device management system in accordance with some aspects of the disclosure

FIG. 9 is a diagram illustrating an example of a system for implementing certain aspects of the present technology.

### DETAILED DESCRIPTION

Certain aspects of this disclosure are provided below. Some of these aspects may be applied independently and some of them may be applied in combination as would be apparent to those of skill in the art. In the following description, for the purposes of explanation, specific details are set forth in order to provide a thorough understanding of aspects of the application. However, it will be apparent that various aspects may be practiced without these specific details. The figures and descriptions are not intended to be restrictive.

The ensuing description provides example aspects only and is not intended to limit the scope, applicability, or configuration of the disclosure. Rather, the ensuing description of the example aspects will provide those skilled in the art with an enabling description for implementing an example aspect. It should be understood that various changes may be made in the function and arrangement of elements without departing from the spirit and scope of the application as set forth in the appended claims.

The terms “exemplary” and/or “example” are used herein to mean “serving as an example, instance, or illustration.” Any aspect described herein as “exemplary” and/or “example” is not necessarily to be construed as preferred or advantageous over other aspects. Likewise, the term “aspects of the disclosure” does not require that all aspects of the disclosure include the discussed feature, advantage or mode of operation.

As used herein, the term “consignment” or “consigned device” means any device or asset not owned by the facility or not involved in a sale. The ownership of a consigned device does not change.

As previously described, the management of equipment may be a challenge for various entities due to various constraints and core competencies. In some cases, facilities may have difficulty in maintaining equipment in a ready state due to labor challenges, difficulty in maintaining an inventory, and difficulty in maintaining consigned devices. These challenges can create problems within a medical facility.

Disclosed herein are systems, methods, and computer-readable storage media for managing devices in various settings. In some aspects, the approaches herein can complement and/or integrate into a facility's operations and improve consigned device visibility. The device management system is a cloud-based system that can be provided to various client devices without incurring any infrastructure costs (e.g., a web-based application) and improve the operational flow of medical or scientific facilities.

Additional details and aspects of the present disclosure are described in more detail below with respect to the figures.

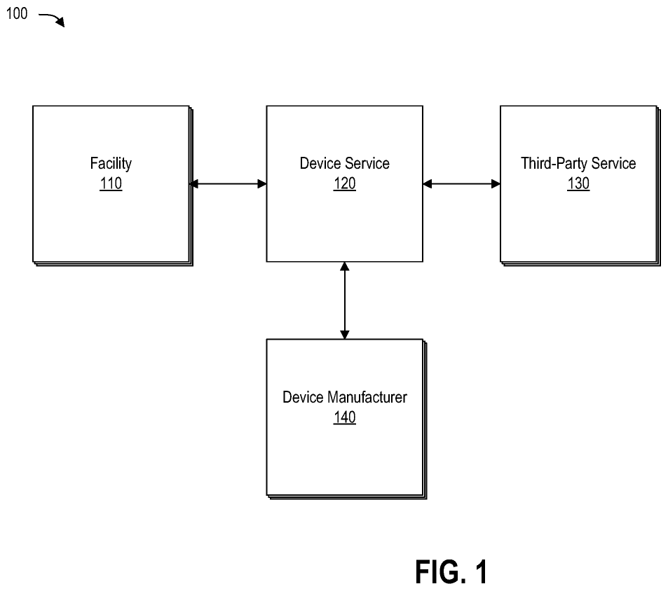

FIG. 1 is a conceptual diagram 100 illustrating a device management system, in accordance with some aspects of the disclosure. In some aspects, the device management system includes at least one facility 110, a device service 120, at least one third-party service 130, and at least one device manufacturer 140. In some aspects, each facility 110 employs the device service 120 to assist in the management of one or more devices that may be used by the facility 110. In some aspects, each facility 110 has a core function to a primary clientele, and various operations of the devices, equipment, and instruments needed to perform those core functions. An illustrative example of a facility 110 is a medical facility with provides medical care to individuals, but requires medical equipment, devices, and instruments to perform those functions. Similar to many businesses, a medical facility may have dynamic requirements and therefore use various methods to manage and maintain its infrastructure. In this case, infrastructure can be medical devices, equipment, and instruments, but can also include other tertiary systems such as network equipment, servers, billing systems, a patient management system, sterilization systems, and so forth. In many cases, some of the infrastructure is fixed and is foundational to the operation of the facility such as a billing system, a patient management system, and various medical devices. Other aspects of the infrastructure are dynamic, such as various medical instruments that are required on an infrequent basis or a rotating basis.

According to aspects of the disclosure, the facility 110 may engage a third-party service 130 to provide consigned items on a temporary basis and internally manage the consigned items. In some illustrative aspects, the consigned items may be devices that are specialized for a limited number of operations and cost prohibitive for a facility 110 to acquire these items. In other cases, the consigned items may be related to the preferences of a particular operator (e.g., a surgeon, etc.). For example, a surgeon that is on call or assigned to the surgery may have particular preferences for various instruments and may require these items to be prepared by the facility 110. In other cases, the consigned items may be related to the specific surgery to be performed on the patient. For example, a scheduled surgery for a specific patient may have a need for a particular set of instruments and may require these items to be prepared by the facility 110.

Although the aspects described above describe the facility 110 as a medical facility, the facility 110 can be any type of research or development facility that has a dynamic and continually rotating need for different equipment, tools, instruments, or devices. For example, a university may require a specific type of equipment as part of an experiment.

According to aspects of the disclosure, the device service 120 may provide a consignment service to facilitate consignment, sanitation, repair, decontamination, maintenance, and other operations associated with the infrastructure of the facility 110 or use of the consigned items in conjunction with the third-party service 130 and the device manufacturer 140. The device service 120 may provide infrastructure assets to the facility 110 to assist coordination of the various infrastructure operations and device use and implement data recording processes to identify operational deficiencies within the facility 110. The device service 120 may also reduce operation overhead and infrastructure requirements to enable the facility 110 to provide more attention to core tasks related to the operation of the facility 110. For example, a conventional facility 110 manages consigned and owned infrastructure, and the staff of the facility 110 must coordinate delivery, inventory, sterilization, return, repair, and other tertiary tasks. The large number of tertiary tasks performed by the facility 110 induce stress on top of bureaucratic inefficiencies (e.g., legal requirement, insurance, etc.) and reduce the attention that staff can provide to more meaningful operations, such as patient management, billing, and so forth. In some aspects, the device service 120 is a cloud-based service that provides one or more applications to various parties as further described herein.

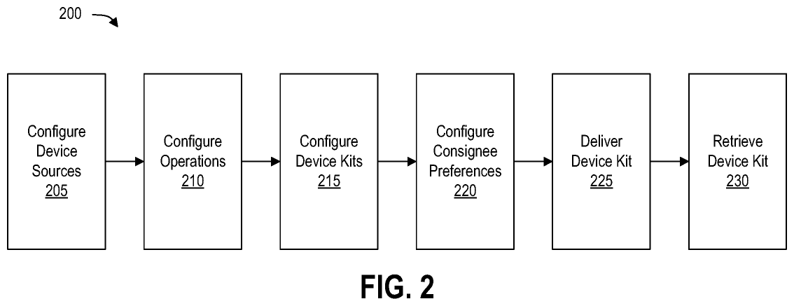

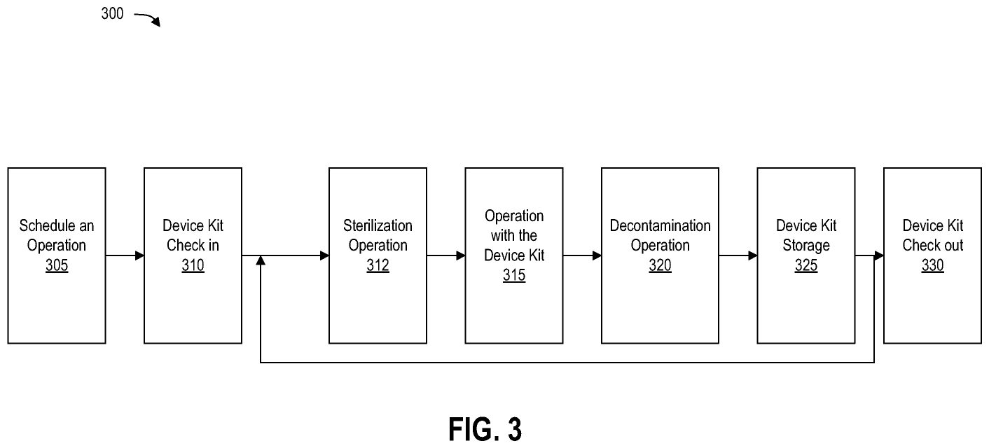

According to some aspects, the device service 120 includes various components to coordinate at least one third-party service 130 and at least one device manufacturer 140 to provide operation and logistical information to the device service 120 to improve operations at the facility 110. Based on input data provided by the facility 110, the device service 120, and the third-party service 130, the device service 120 can vertically align supply chain logistics, educational opportunities, maintenance, sterilization, consignment, and other information using a standardized interface to simplify operations between multiple interested entities. An example of operations of the third-party service 130 is further described with reference to FIG. 2 below, and an example of operations of the facility 110 is further described with reference to FIG. 3 below. The described operations enable various operators, staff, and agents that employ the device service 120 to coordinate more efficiently by removing extra steps in conventional processes. In addition, the device service 120 is configured to track data that is provided as part of operations within the facility 110 to identify operational deficiencies, coordinate maintenance, coordinate surgeries, assist in training operations, and so forth. The device service 120 can configure the operational aspects at the facility 110 to remove private and patient information from any data to comply with regulatory and/or privacy requirements.

In some cases, the device service 120 may also coordinate with at least one device manufacturer 140. For example, operational data provided by the facility 110 or the device manufacturer 140 can provide education data (e.g., educational opportunities, training, recommendations, etc.) to the device service 120, which is then made available to the facility 110 through the device service 120.

FIG. 2 is a conceptual flow diagram 200 illustrating an example of operations performed by a third-party service (e.g., the third-party service 130) associated with a device management system in accordance with some aspects of the disclosure. The device service 120 may provide infrastructure operations to (e.g., the facility 110) in conjunction with a third-party service (e.g., the third-party service 130) and a device manufacturer (e.g., the device manufacturer 140). In one illustrative aspect, the device service 120 may be configured to provide a cloud-based application that connects various endpoints, which enables the separation of concerns as described above. For example, the device service 120 may distribute an application using various techniques. Examples of application distribution techniques include a native application (e.g., having native processor instruction) that is distributed using a store, a downloadable application having native processor instructions or interpretable instructions (e.g., an application using the Electron framework), a cloud-based application capable of being executed in a browser sandbox (e.g., a JavaScript application, a webassembly application), or a server-based application that is rendered at a server that is provided to a client application.

In some cases, the device service 120 is configured to shift operational aspects of the consignment to the third-party service to allow the facility 110 to perform core functions. In some cases, this type of function separation is referred to as the separation of concerns to enable parties, functions, and/or services to consolidate responsibility for an aspect of their domain, which enables operational efficiency and decreases attention dimensions. Attention dimensions are the number of parameters that a party needs to consider during various processes and limiting the number of dimensions improves the ability to provide attention to core functionality. For example, by enabling a third-party service to identify one or more device kits to the device service 120, which is a set of devices, equipment, instruments, and/or accessories, the facility 110 does not need to separately manage the device kits within an internal system. In one illustrative aspect, the device kit may be a medical kit, which may also be referred to as a medical tray or similar. In this case, the device kit is not a physical tray, but a collection of items that may be referred to as a tray because one or more instruments may be physically delivered on a tray. However, the device kit can include carts, trays, self-standing equipment, test equipment, measurement equipment, and so forth. The device kit can also be related to semiconductors, chemicals, electrical, construction, or other fields. For example, the device kit may be a scanning electron microscope (SEM), a semiconductor wafer probe, or other specialized component that a facility may require.

In one aspect, the third-party service may configure device sources of the device management system at block 205. An example of a device source is a device manufacturer, and the third-party service procures items directly from the device manufacturer or a representative of the device manufacturer. In some examples, the third-party service is a device manufacturer representative or vendor. The third-party service may represent more than one device manufacturer.

The third-party service may also configure operations at block 210 for the device management system. In some cases, the operations are various tasks that may be associated with a facility and are associated with a core competency. For example, a medical facility may be associated with different medical operations. In some cases, the operations can be defined within the device management system and the third-party service can supplement the operations based on various types of equipment that are provided by one or more device manufacturers to the third-party service. The third-party service can be trained to interact with the facility and may have knowledge of operational parameters and therefore can configure one or more operations based on one or more device kits available from the third-party service. In this manner, the third-party service enters information relevant to services, devices, and products into the device management system that corresponds to their core competency.

At block 215, the third-party service may configure one or more device kits within the device management system at block 215 and may be associated one or more device kits with operations that are known to the device management system. In the case of a medical facility, the operation may be a medical operation, part of a study, or other scientific operation. The operation may be different based on the core functionality of the facility, such as a hospital, surgery center, university, a chemical manufacturer, a laboratory, etc.

The third-party service may also configure consignee preferences at block 220. For example, a particular operation (e.g., a surgeon or other medical professional), may have a particular preference associated with a device kit. In this case, the third-party service may specify a device kit and create multiple versions of the same device kit using, for example, value equality (e.g., whether the data is a pointer to another location or a full plain object).

At block 225, the third-party service may deliver a device kit to a facility for usage. For example, the third-party service may offer the device kit for consignment to medical facilities to ensure that the device kit is being efficiently allocated for use. In some cases, the third-party service may use an application to capture evidence (e.g., screenshots) of a device kit that is being delivered. The evidence can be geotagged to enable location data to provide evidence of such delivery.

At block 230, the third-party service may retrieve the device kit. For example, the facility may return the device kit because an operation is completed and does not require the device kit. In other examples, the device kit may require servicing as part of its normal operations. For example, a device may need to be serviced after being used in a set number (e.g. 5) of different operations, and a third-party service may need to facilitate service with a corresponding device manufacturer. In other cases, the device kit may be damaged or otherwise require maintenance and the facility may initiate a maintenance operation.

In this manner, the device management system maximizes usage, maintenance, and operation of the device kit using a platform that allows each party to focus on a core competency.

FIG. 3 is a conceptual flow diagram 300 illustrating an example of operations performed by a facility associated with a device management system in accordance with some aspects of the disclosure. In some cases, the device service 120 may provide infrastructure operations to the facility (e.g., the facility 110) in conjunction with a third-party service (e.g., the third-party service 130) and a device manufacturer (e.g., the device manufacturer 140). In one illustrative aspect, the device service 120 may be configured to provide a cloud-based application that connects various endpoints, which enables the separation of concerns as described above. For example, the device service 120 may distribute an application using various techniques. Examples of application distribution techniques include a native application (e.g., having native processor instruction) that is distributed using a store, a downloadable application having native processor instructions or interpretable instructions (e.g., an application using the Electron framework), a cloud-based application capable of being executed in a browser sandbox (e.g., a JavaScript application, a webassembly application), or a server-based application that is rendered at a server that is provided to a client application.

In some aspects, the facility may schedule an operation that includes at least one device kit at block 305. In one example, a person associated with the facility may schedule an operation such as a surgery. In the case of a medical facility, the scheduling operation may be done with a facility management system that can interact with the device management system. In some aspects, a person within the facility may also schedule the operation using an application provided by the device management system (e.g., the device service 120). When the operation is scheduled at block 305 using a corresponding system, the system may be configured to allow the user to identify the infrastructure necessary for the corresponding operation and either reserve or procure the infrastructure. For example, a staff member of a medical facility may schedule an operation associated with a patient, and the application provided by the device management system may identify that a particular medical device kit is necessary. That particular medical device kit can be provided by a third-party service for consignment due to the facility not being able to access it. For example, the medical facility can link an internal system to the device management system to enable the device management system to identify the status of the various device kits. In other cases, the device management system may manage the inventory of the facility. The staff member can then take the necessary steps to procure the device kit from the third-party service. In this case, the device management system can provide information related to the device kit to a third-party service to coordinate delivery of the device kit in connection with the scheduled operation.

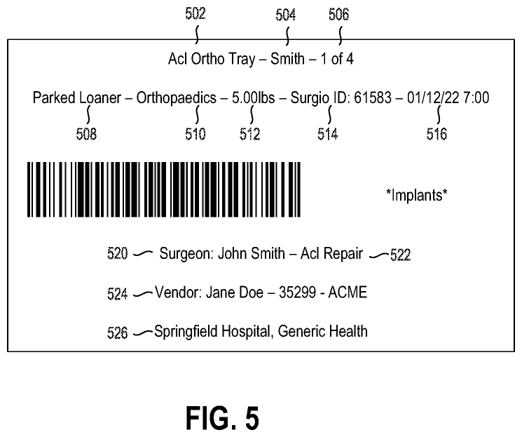

At block 310, the facility or the third-party service can perform a check-in process associated with a device kit upon delivery based on the device management system. For example, the device management system is a cloud-based application and a user at the facility registers that the device kit is successfully delivered. In some aspects, the user at the facility is a user associated with the third-party service that is physically at the facility. This allows a single user to check-in the device kit without disrupting the facility. Typically, the user checking in the device kit has the most information about the devices and the operation it is intended for at the time of check-in. Allowing a user from the third-party service perform the check-in of the device kit can improve efficiency and accuracy of the information provided at check-in. In one aspect, the user registers the device kit and generates a tag to provide on the device kit. One illustrative example of a tag is a label that is coded with various information provided by the facility and/or manufacturer. For example, the label may have an identification of an operation, an operator (e.g., the medical professional), information pertaining to the operation, information pertaining to the devices in the device kit, and encoded binary information that links to the operation (e.g. a barcode). An example of a label is further illustrated with reference to FIG. 5. Other examples of tags include a radio frequency identifier (RFID) tag, or a wireless tracker (e.g., a Bluetooth™ tracker, etc.) that can link the physical placement and operation of the device kit to the device management system.

At block 312, the device may enter a sterilization operation (e.g., maintenance operation). The device may be sterilized in a variety of ways including steam cleaning, dry heat cleaning, radiation, ethylene oxide gas, vaporized hydrogen peroxide, and other sterilization methods known in the art. Additionally, the device may be sterilized before the device check in step and then sterilized again at block 312. In some examples, block 310 and 312 may be switched, such that the device is sterilized before it is checked in.

At block 315, an operation with a device kit at the facility is performed. For example, the device kit may be used in surgery. In this case, block 315 may also include various pre-processing operations and post-processing operations at the facility. For example, a pre-processing step may be to verify the operation of the device using a standardized checklist. In some cases, the standardized checklist can be provided by the device manufacturer and may be input into one or more systems to record requisite information for regulatory or legal purposes. An example post-processing operation can be similar to the pre-processing operation and ensure that no physical damage to the device kit, including various accessories associated with the device kit, occurred and the device kit is not compromised. In some cases, the pre-processing operation and the post-processing operation may provide a status to the device management system to indicate the device kit has been used and is entering another state. For example, when the label of the device kit is scanned during pre-processing or post-processing at block 315, the device management system can provide store information pertaining to the state change (e.g., entering the operation room (OR), or leaving the OR, etc.). This information can be used by the device management system to identify operational deficiencies that are individually impossible to see at the macro level due to the disaggregated nature of the systems and techniques used within the facility.

At block 320, the device kit may enter a decontamination operation (e.g., a maintenance operation associated with the facility). In one aspect, the device kit may be provided to a sterile processing department (SPD) after the operation at block 315. Similar to the operation, the device kit may undergo a pre-processing step such as scanning the tag to register that the device kit is unavailable for use until processing is complete. In some aspects, the scanning of the tag may be used to track the performance of the SPD and/or individuals within the SPD to identify operational deficiencies.

At block 325, the device kit may enter into a storage stage in which the device kit is placed into a holding area until needed again. In some cases, the device kit may be removed from the storage stage and provided to a sterilization operation at block 312 before being provided for another operation at block 315. In other cases, a portion of the devices in the device kit may be requested for an operation, and the portion of the devices may be selectively redirected to another operation. In such cases, the device management system may require all or part of the device kit. In the event that part of the device kit is requested for an operation, the device management system is configured to enable tracking of the device kit by adding a new device kit that is a subset of the original device kit and may link the subset to the original in order to return the subset to merge back into the device kit.

At block 330, the device kit may be returned to the third-party service. Similar to block 310, the facility may perform a checkout process to return the device kit to the third-party service. As noted above, the tag enables a department to check in/check out device kits to identify the status of the device kit and track its location, state, and status. In some cases, the tag may also include geo-location capabilities such as an Apple AirTag™ or similar technology to enable location servicing.

In some aspects, blocks 312, 315, 320, and 325 may be repeated to conduct two operations before the device kit is checked out. In another example, blocks 312, 315, 320, and 325 may be repeated 1, 2, 3, 4, 5, 6, 7, 8, 9, 10, or more times before the device kit is checked out at block 330.

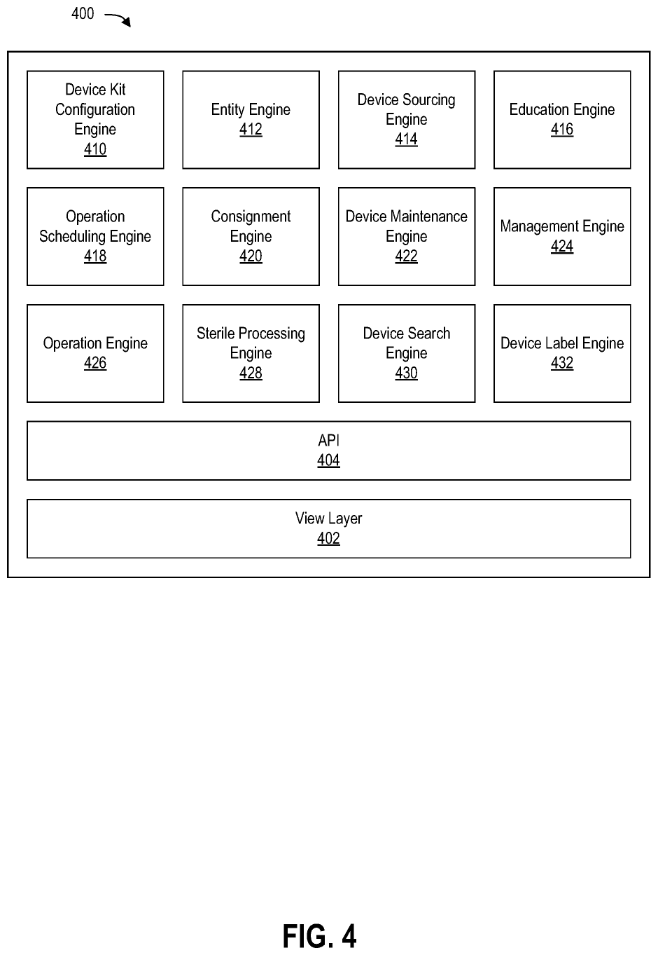

FIG. 4 is a functional block diagram 400 of a device management system in accordance with some aspects of the disclosure and includes a number of components to enable the operation of the device management system. In one illustrative aspect, the device management system includes a view layer 402 that is configured to render the view based on the various components of the device management system. In some aspects, the view layer 402 can be configured to render the view at the server or at the client. For example, a client-rendered device management system sends instructions to a client application and the client application uses a separate client state to determine how to render the view (e.g., markup). In a server-rendered application, a client state is stored at the server and the server renders the view and sends the rendered view to the client.

In some aspects, the device management system also includes an application programming interface (API) that can expose functionality to one or more client applications. For example, a client-rendered device management system uses an API to send and receive data (e.g., a list of operations, a list of facilities, etc.) and render a state at the client based on the requested data.

The device management system includes a plurality of engines that are configured to perform various functions. In some cases, each engine can be an endpoint associated with the API 404 or may be implemented in a manner to separate concerns and prevent duplicate functions and unintended consequences. In one aspect, the device management system includes a device kit configuration engine 410 for configuring various device kits (e.g., by the third-party service, by a facility, etc.), an entity engine 412 for managing relationships (e.g., with a device manufacturer), a device sourcing engine 414 to sourcing supplies with the various entities, an education engine 416 to interfacing customers and/or agents with a manufacturer for learning, training, and other opportunities. In one illustrative aspect, the education engine 416 can link to the manufacturer for various information, such as maintenance, training functions, videos, and other information that may be useful for the facility.

The device management system may also include an operation scheduling engine 418, for scheduling an operation or retrieving information related to a scheduled operation, a consignment engine 420 or various consignment operations described with respect to FIG. 2 and FIG. 3, a device maintenance engine 422 related to maintenance operations, a management engine 424 related to the management of various device kits, an operation engine 426 related to determining operation efficiency of the facility (e.g., internal processing such as delivery and retrieval of device kits), a sterile processing engine 428 related to determining sterilization operations of the facility, a device search engine 430 for searching and identifying devices and instruments within the facility, and a device label engine 432 for generating labels for the device kits. In some aspects, the operation engine 426 may also be used to analyze previous operations. In one aspect, a user may request an analysis of an operation type from the medical instrument service and, in response, receive previous information related to the operation type from the medical information. In one illustrative example, the device management system may surface information related to previous operations such as conditions that led to the failure of one or more devices. In some cases, the previous information includes the identification of a configuration of each operation type, and wherein the configuration identifies at least a set of medical devices.

In some cases, each of the various engines described above may be configured in a self-contained unit (e.g., a class, a module, etc.) and configured to provide data (e.g., objects, interfaces, types, structs) that correspond to the engine to efficiently separate functions, API calls, and so forth. In general, the various engines are separated from the view layer 402 to separate the concerns of the view layer 402 from the domain (e.g., the various engines 410 to 432). Other modules and engines may also be included such as an authentication module, and a database repository layer, and are omitted for clarity.

FIG. 5 illustrates an example label 500 that is provided by a device management system in accordance with some aspects of the disclosure. The example label includes various information such as a name 502 that identifies the device kit, a type 504 of the device kit, and a count 506 that identifies the number of components within the device kit. In this case, the device kit is a medical device kit and the type 504 corresponds to a particular configuration of the device kit for a particular surgeon. The label 500 also includes information such as the parked loaner status 508, a department 510, a weight 512, a medical kit identifier 514 associated with the device management system, and a date 516. The label may be produced or provided by a printer or other machine capable of producing the label.

In some aspects, the label 500 includes a barcode 518 or other machine readable label (e.g., an RFID tag, etc.) that links a device that reads the label to the device management system. In some cases, the barcode 518 can be used to check in the device kit into an OR as part of a preparation process for the operation or check out the device kit from the OR to indicate that the device kit is released from the OR. The barcode 518 can also be used to check in and check out the device within various operations associated with the facility (e.g., sterile processing, short-term storage, etc.). The barcode may store and send information related to the location of the device kit. The device kit may be checked into or out of an operation by scanning the barcode. For example, when the device kit is checked into or out of an operation (e.g., transit, consignment, sterilization operation, medical operation, decontamination operation, and/or storage), the barcode may send information to a server or computing system. In this manner, the location and status of the device is tracked and may be accessed by a user on the computing system or server at any time.

The label 500 may be generated during a facility check-in process (e.g., corresponding to block 310 in FIG. 3) and attached to each component associated with the device kit. For example, a device kit can be medical equipment that includes a number of accessories (e.g., tubes, connectors, probes, etc.) that may also be provided as part of the device kit. The label 500 may also include an identity 520 of the person performing operation 522 to assist in routing the device kit to the correction location within the facility. In some cases, the location of the operation may not be provided on the label 500 because the location of the operation is determined at a time closer to the operation. In that case, a user of the device management system may scan the barcode 518 to determine where to route the medical it. As illustrated in FIG. 5, label 500 can include identifying the third-party service 524 and a facility 526.

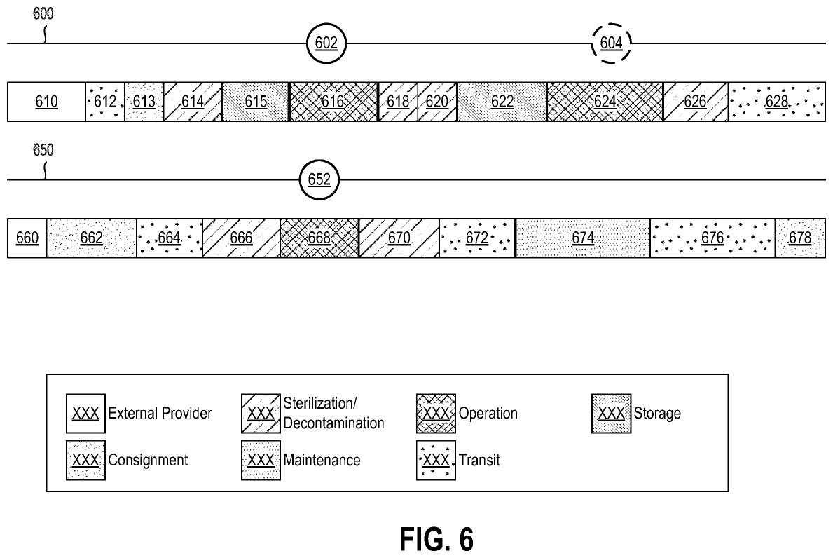

FIG. 6 illustrates various timelines that describe the operation of a device management system in connection with a medical facility in accordance with some aspects of the disclosure. In one aspect, a first timeline 600 is related to a first operation 602 of a patient and a second operation 604 of a patient. In some examples, the second operation 604 may be a standby operation associated with a medical professional. In one aspect, the third-party service may have possession of a device kit at block 610 and a facility may request consignment of the device kit. At block 612, the device kit may be in transit to the facility. At block 613 (i.e., consignment), the third-party service may provide the device kit to the facility and perform various operations associated with the device kit. For example, the facility may perform a check-in process and generate a label (e.g., the label 500 in FIG. 5), which registers the status of the device kit in the device management system. A sterilization process may begin at block 614. For example, the device kit is provided to an SPD associated with the facility. As noted above, the device kit can be checked into and out of the SPD using the label and the device management system. At block 615, the device kit is in storage until the device kit is provided to operation 616. As noted above, operation 616 can include a check-in and check-out process that is performed in conjunction with the device management system. At block 618, the device kit enters a decontamination process after the operation 616 is completed. For example, the device kit is provided to an SPD associated with the facility. As noted above, the device kit can be checked into and out of the SPD using the label and the device management system. The decontamination process may be any decontamination process known in the art and the sterilization process may be any sterilization process known in the art. At block 620, the device kit enters a sterilization process. For example, the device kit is provided to an SPD associated with the facility. As noted above, the device kit can be checked into and out of the SPD using the label and the device management system. When decontamination and sterilization occur sequentially, the device kit may remain checked into the SPD for both processes. In other examples, the decontamination process may take place in a different location within the facility than the sterilization process. In this example, the device kit may be checked into the decontamination location, decontaminated, checked out of the decontamination location, and then checked into the sterilization location (SPD). After the sterilization process at block 620. the device kit may be checked into storage at block 622.

In some aspects, the device kit may be associated with a second operation 604. In some examples, the second operation 604 may be a standby operation of a particular medical professional, who is on call for a duration. The device kit is checked out from storage and is transited to operation 624. As noted above, operation 624 can include a check-in and check-out process that is performed in conjunction with the device management system. In some examples, the device kit is utilized in the operation 624. In other examples, the device kit may not be used in operation 624. If the device is utilized, the device kit is provided to a decontamination process 626. For example, the device kit is provided to an SPD associated with the facility. As noted above, the device kit can be checked into and out of an SPD associated with the facility. At block 628, the device kit is in transit back to the third-party service. In the case where the medical professional does not utilize the device kit at operation 624, the decontamination process 626 may be skipped. In some examples, before the device kit is in transit at block 628, the device kit undergoes a consignment operation at the facility to check the device kit out of the facility. Once the device kit is returned to the third-party service, the third-party service may perform various operations (e.g., maintenance, etc.) of the device kit.

In some aspects, the facility uses the device management system at various operational stages and maintains an accurate inventory and tracking system on behalf of the facility, while minimizing operations associated with or required at the facility.

A second timeline 650 illustrates a similar process but includes a maintenance operation that can occur in conjunction with the device management system and the third-party service. In one aspect, a first operation 652 is scheduled for a device kit. In this case, the third-party service is in possession of the device kit at block 660, the third-party service delivers the device kit to the facility for a consignment operation at block 662. At block 664, the device kit is transited to a sterilization process 666. For example, the device kit is provided to an SPD associated with the facility. As noted above, the device kit can be checked into and out of an SPD associated with the facility. The device is then provided to operation 668, corresponding to the first operation 652. In some aspects, the device kit may become damaged due to one or more issues. For example, during check-out of the operation 668, the device kit may fail a check-out operation, indicating some malfunction. In other examples, a calibration may be invalidated, or a part needs to be replaced after a maximum number of uses.

In some cases, at operation 668, the third-party service is provided information related to the malfunction and the third-party service is assigned an operation to retrieve the device kit for maintenance (e.g., repair). The device kit is decontaminated during decontamination process 670. At block 672, the device kit is transited by the third-party service to maintenance. In some examples, the device kit will be checked out of the facility at a consignment operation before being transited by the third-party service to maintenance. At block 674, a maintenance operation is performed (e.g., by the third-party service). In some examples, maintenance may include repair of one or more components of the device kit. After maintenance, the third party service may transit the device kit back to the facility at block 676 for a consignment operation at block 678. After consignment at block 678, a second operation may occur in substantially the same manner as the first operation. In other examples, a second operation may occur with one or more additional blocks or less blocks than the first operation. For example, after the device kit is returned to the consignment operation at block 678, the device may undergo a sterilization process, followed by a second operation, a decontamination process, and then a consignment process where the third-party provider retrieves the device from the facility. After the device kit is retrieved, it may be in transit back to the third-party provider. In this manner, the device management system enables efficient operation of the facility and the third-party service by ensuring efficient communication and alignment of interests. In some cases, the third-party service is interested in ensuring that the device kit is repaired in time for the second operation to provide higher quality service for the facility, and the facility would prefer to not reschedule the second operation.

In some aspects, the device kit may be used for only one operation before being returned to consignment and thereby the third party service. It will be appreciated that other combinations and orders of the blocks in FIG. 6 may be used. For example, transit within the facility is not shown in the first timeline 600, but transit may occur within the facility between different blocks. In some examples, the device kit may be checked in and out using the device management system at every block or some of the blocks.

The first timeline 600 and the second timeline 650 are illustrative examples that describe how the device management system enables efficient asset resource allocation using the principles of separation of concern, and alignment of interests using a single view layer. In some cases, however, the view layer can be integrated into other systems using various known mechanisms. For example, in some application architectures, the view layer can be provided through another application view. For example, the view layer can be server-rendered by the device management system, and the view can be provided in a different application based on the different applications and the device management system coordinating the view. For example, a server-rendered framework (e.g., SolidJS, React Server Components, Blazor Server, etc.) can be passed through another application using an iframe or other isolation technology. In some cases, the device management system may be a native or interpreted application that has a webview render layer that renders an application based on an embedded hypertext markup language (HTML) component (e.g., Microsoft Edge™, WebKit, etc.).

In some aspects, each of the facility and the third-party service use the device management system to coordinate timely delivery, return, and maintenance of devices by the third-party service. The efficiencies enable the facility to focus on core competencies and reduce the attention requirements, simplify operations, and allow a facility to focus on core competencies rather than administrative logistics.

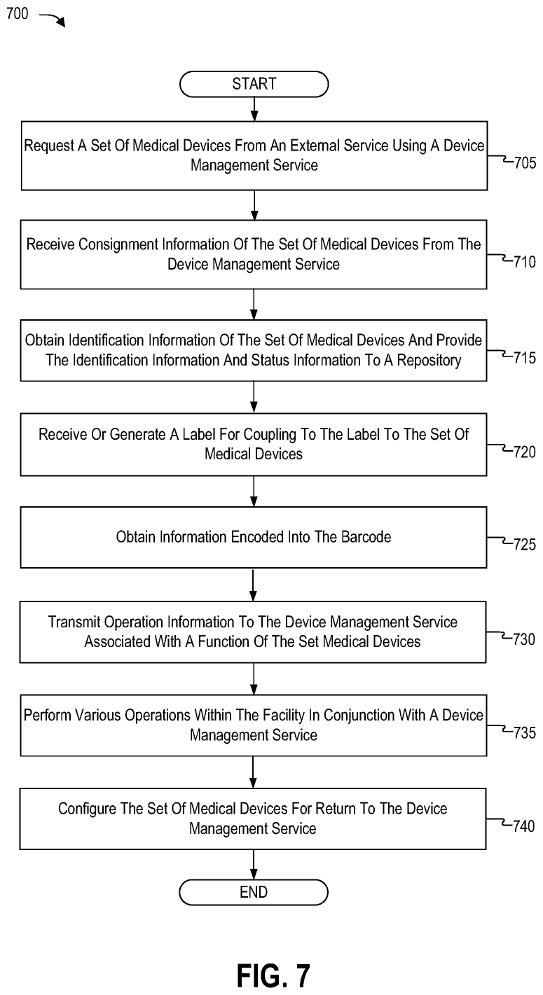

FIG. 7 illustrates an example of method 700 of a cognition management system in accordance with some aspects of the disclosure. The method 700 can be performed by a computing device having network communication capabilities and may be configured as a service separate from a client system, or may be integrated into the client system. For instance, the computing system 1300 may be configured to perform all or part of the method 700.

Although the example method 700 depicts a particular sequence of operations, the sequence may be altered without departing from the scope of the present disclosure. For example, some of the operations depicted may be performed in parallel or in a different sequence that does not materially affect the function of the method 700. In other examples, different components of an example device or system that implements the method 700 may perform functions at substantially the same time or in a specific sequence.

At block 705, the computing system may request a set of medical devices from an external third-party service using a device management service. For example, the external service may be a third-party vendor associated with a device manufacturer, or may be a third-party vendor having an inventory of device kits and is independent of any device manufacturer.

At block 710, the computing system may receive consignment information of the set of medical devices from the device management service. In one aspect, the external service provides information to the device management service to trigger the consignment information.

At block 715, the computing system may obtain identification information for the set of medical devices and provide the identification information and status information to a repository. For example, block 715 may occur during consignment when a third-party service delivers a set of medical devices. The third-party service may host the repository, but the repository could also be a cloud-based repository, database, or other storage mechanism (e.g., object storage, block storage).

At block 720. For example, the computing system may receive or generate a label for coupling the label to the set of medical devices. The label comprises medical device information and a barcode with identifier information. The medical device information comprises the identification of medical events and at least one person associated with the event. The status of the set of medical devices is changed in the device management service in response to providing the label to the computing device.

At block 725. For example, the computing may obtain information encoded into the barcode. In some aspects, the obtaining of the information may occur based on a status change of the set of medical devices. For example, the obtaining information encoded into the barcode can happen during check-in or check-out of an operation in an OR, check-in or check-out of a sterilization process, etc.

At block 730, the computing system may transmit operation information to the device management service associated with a function of the set medical devices. The device management service updates the status of the set of medical devices. The device management service updates the status of the set of medical devices based on information included in the operation information.

At block 735, the computing system may perform various operations within the facility in conjunction with a device management service. Different examples of the various operation are further described below.

In one example of block 735, the computing system may transmit a query requesting identification of a first medical device within a facility associated with the computing device, and receive identification of a second set of medical devices within the facility including the first medical device. For example, a medical professional of the facility may require a particular instrument, and a user may input information pertaining to one or more items, and the computing system may transmit the query to identify any potential source known to the device management system.

In another example of block 735, the computing system may request status information from the device management service pertaining to at least one status associated with sets of medical devices within a facility associated with the computing device. For example, the computing system may request a performance metric associated with one or more statuses of devices (e.g., the set of devices is in sterile processing, the set of devices is undergoing maintenance, etc.). In response, the computing system may receive status information identifying performance of the at least one status within the facility. The status information includes performance metrics associated with at least one of the sterilization operations, consignment operations, maintenance operations, or internal operations within the facility.

In another example of block 735, the computing system may display information related to a current operation associated with the set of medical devices. In one aspect, the computing system may request the current status of consigned items and the list may include at least a portion of the current consigned items with status information (e.g., in sterile processing, available, unavailable, etc.).

The computing system may request an analysis of an operation type from the medical instrument service in block 735. The previous information comprises information identifying at least one medical operator. The analysis of the operation type includes a parameter associated with at least one of a patient, a medical condition, or an operation type.

In another example of block 735, the computing system may receive previous information related to the operation type from the medical information. In this example, the computing system may request information pertaining to the operation type and receive information regarding the previous operation. In this case, the information regarding the previous operation can be modified to omit patient identifying information, but list relevant information for the medical professionals' guidance. The previous information includes identification of a configuration of each operation type. The configuration identifies at least set of medical devices.

In another example of block 735, the computing system may transmit servicing information to the medical instrument service related to servicing the set of medical devices. The servicing information is provided to the external service to trigger at least one operation for at least one medical device of the set of medical devices. For example, the at least one medical device may be compromised during a previous operation and may need to be serviced or replaced by the third-party service.

At block 740, the computing system may configure the set of medical devices to return to the device management service. In this case, the computing system can receive the set of medical devices to return them from the consignment.

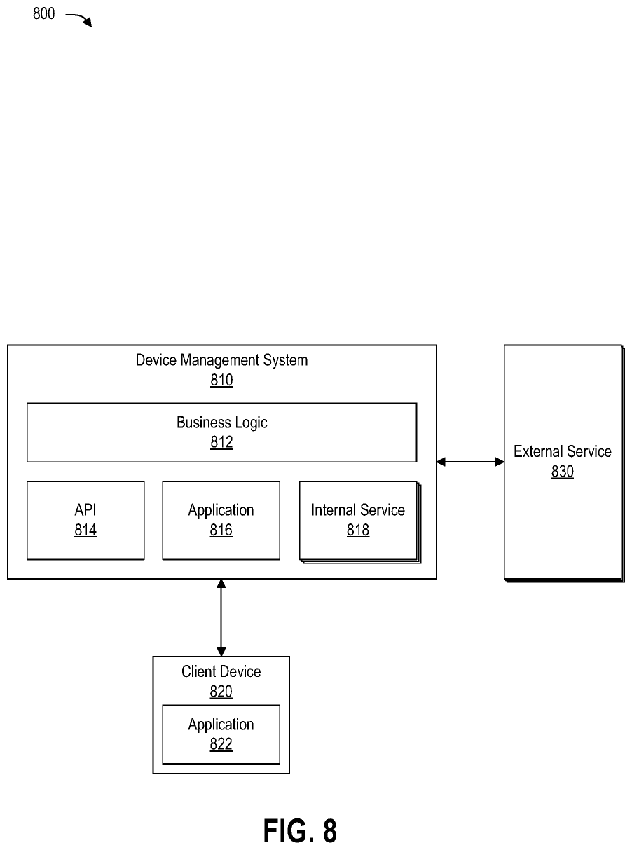

FIG. 8 is a conceptual diagram 800 illustrating an architecture and deployment of a device management system 810 in accordance with some aspects of the disclosure. The device management system 810 is configured to provide application functionality to a client device 820 using various techniques and may employ one or more external services 830. In accordance with some aspects, the device management system 810 includes a business logic 812 to perform various domain-specific functions of the device management system 810. For example, the device management system 810 may collect information from the external services 830, analyze unstructured data, and so forth. The device management system 810 may also include an API 814, an application 816, and one or more internal services 818.

The API 814 may be employed in some scenarios such as when a client device 820 is executing an application 822 natively (e.g., without the use of an interpreter). In some cases, the application 816 may be sent to the client device and the application 822 executes the application 816 in a sandbox. Examples of sandboxed applications include various web applications that use client-side rendering techniques such as React, Angular, SolidJS, Blazor, and so forth. Sandboxed applications may use the API 814 to send requests to the device management system 810, and the business logic 812 generates a response, which is provided to the client device 820.

In some cases, the application 816 may be executed within the device management system 810 to provide a view, which is the state of the current render, to the client device 820. In some cases, the API 814, the application 816, or the application 816 may engage with the internal services 818 for various purposes. The internal services 818 can be, for example, various containerized microservices or another type of distributed architecture.

The external services 830 are used for various purposes, such as to collect data from various sources. As described above, the sources can be related to user manuals, research papers, standards proposals, and so forth. In other cases, the external services can be storage services (e.g., object storage, block storage, etc.), monitoring services, authentication services, etc.

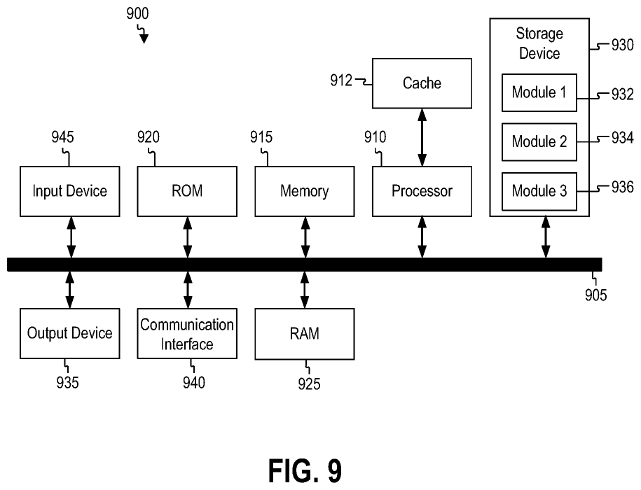

FIG. 9 is a diagram illustrating an example of a system for implementing certain aspects of the present technology. In particular, FIG. 9 illustrates an example of computing system 900, which can be for example any computing device making up an internal computing system, a remote computing system, a camera, or any component thereof in which the components of the system are in communication with each other using connection 905. Connection 905 can be a physical connection using a bus, or a direct connection into processor 910, such as in a chipset architecture. Connection 905 can also be a virtual connection, networked connection, or logical connection.

In some aspects, computing system 900 is a distributed system in which the functions described in this disclosure can be distributed within a datacenter, multiple data centers, a peer network, etc. In some aspects, one or more of the described system components represents many such components each performing some or all of the function for which the component is described. In some aspects, the components can be physical or virtual devices.

Example computing system 900 includes at least one processing unit (CPU or processor) 910 and connection 905 that couples various system components including system memory 915, such as ROM 920 and RAM 925 to processor 910. Computing system 900 can include a cache 912 of high-speed memory connected directly with, in close proximity to, or integrated as part of processor 910.

Processor 910 can include any general purpose processor and a hardware service or software service, such as services 932, 934, and 936 stored in storage device 930, configured to control processor 910 as well as a special-purpose processor where software instructions are incorporated into the actual processor design. Processor 910 may essentially be a completely self-contained computing system, containing multiple cores or processors, a bus, memory controller, cache, etc. A multi-core processor may be symmetric or asymmetric.

To enable user interaction, computing system 900 includes an input device 945, which can represent any number of input mechanisms, such as a microphone for speech, a touch-sensitive screen for gesture or graphical input, keyboard, mouse, motion input, speech, etc. Computing system 900 can also include output device 935, which can be one or more of a number of output mechanisms. In some instances, multimodal systems can enable a user to provide multiple types of input/output to communicate with computing system 900. Computing system 900 can include communications interface 940, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Apple® Lightning® port/plug, an Ethernet port/plug, a fiber optic port/plug, a proprietary wired port/plug, a Bluetooth® wireless signal transfer, a BLE wireless signal transfer, an IBEACON® wireless signal transfer, an RFID wireless signal transfer, near-field communications (NFC) wireless signal transfer, dedicated short range communication (DSRC) wireless signal transfer, 802.11 WiFi wireless signal transfer, WLAN signal transfer, Visible Light Communication (VLC), Worldwide Interoperability for Microwave Access (WiMAX), IR communication wireless signal transfer, Public Switched Telephone Network (PSTN) signal transfer, Integrated Services Digital Network (ISDN) signal transfer, 3G/4G/5G/LTE cellular data network wireless signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof. The communications interface 940 may also include one or more Global Navigation Satellite System (GNSS) receivers or transceivers that are used to determine a location of the computing system 900 based on receipt of one or more signals from one or more satellites associated with one or more GNSS systems. GNSS systems include, but are not limited to, the US-based GPS, the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 930 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity module (SIM) card, a mini/micro/nano/pico SIM card, another integrated circuit (IC) chip/card, RAM, static RAM (SRAM), dynamic RAM (DRAM), ROM, programmable read-only memory (PROM), erasable programmable read-only memory (EPROM), electrically erasable programmable read-only memory (EEPROM), flash EPROM (FLASHEPROM), cache memory (L1/L2/L3/L4/L5/L #), resistive random-access memory (RRAM/ReRAM), phase change memory (PCM), spin transfer torque RAM (STT-RAM), another memory chip or cartridge, and/or a combination thereof.

The storage device 930 can include software services, servers, services, etc., that when the code that defines such software is executed by the processor 910, it causes the system to perform a function. In some aspects, a hardware service that performs a particular function can include the software component stored in a computer-readable medium in connection with the necessary hardware components, such as processor 910, connection 905, output device 935, etc., to carry out the function. The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as CD or DVD, flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some cases, the computing device or apparatus may include various components, such as one or more input devices, one or more output devices, one or more processors, one or more microprocessors, one or more microcomputers, one or more cameras, one or more sensors, and/or other component(s) that are configured to carry out the steps of processes described herein. In some examples, the computing device may include a display, one or more network interfaces configured to communicate and/or receive the data, any combination thereof, and/or other component(s). The one or more network interfaces can be configured to communicate and/or receive wired and/or wireless data, including data according to the 3G, 4G, 5G, and/or other cellular standard, data according to the Wi-Fi (802.11x) standards, data according to the Bluetooth™ standard, data according to the IP standard, and/or other types of data.

The components of the computing device can be implemented in circuitry. For example, the components can include and/or can be implemented using electronic circuits or other electronic hardware, which can include one or more programmable electronic circuits (e.g., microprocessors, GPUs, DSPs, CPUs, and/or other suitable electronic circuits), and/or can include and/or be implemented using computer software, firmware, or any combination thereof, to perform the various operations described herein.

In some aspects the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description above to provide a thorough understanding of the aspects and examples provided herein. However, it will be understood by one of ordinary skill in the art that the aspects may be practiced without these specific details. For clarity of explanation, in some instances the present technology may be presented as including individual functional blocks including functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the aspects in unnecessary detail. In other instances, well-known circuits, processes, algorithms, structures, and techniques may be shown without unnecessary detail in order to avoid obscuring the aspects.

Individual aspects may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but may have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices, or other small form factor personal computers, personal digital assistants, rackmount devices, standalone devices, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific aspects thereof, but those skilled in the art will recognize that the application is not limited thereto. Thus, while illustrative aspects of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, aspects can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate aspects, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C. or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the aspects disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as RAM such as synchronous dynamic random access memory (SDRAM), ROM, non-volatile random access memory (NVRAM), EEPROM, flash memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more DSPs, general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

1. A method comprising:
requesting, using a computing device, a set of medical devices from an external service using a medical instrument service;
receiving consignment information of the set of medical devices from the medical instrument service, wherein the external service provides information to the medical instrument service to trigger the consignment information;
when the set of medical devices is received, obtaining identification information of the set of medical devices from the external service and providing the identification information and status information to a repository;
generating, using the computing device, a label for coupling to the label to the set of medical devices, the label comprising medical device information and a barcode encoded with identifier information, wherein the medical device information comprises identification of medical events and at least one person associated with the event, wherein the label is coupled to the set of medical devices, and wherein a status of the set of medical devices is changed in the medical instrument service in response to providing the label to the computing device;
obtaining, using the computing device, information encoded into the barcode;
in response to obtaining the identifier information from the barcode, transmitting, to the medical instrument service, operation information associated with a function of the set of medical devices, wherein the medical instrument service updates the status of the set of medical devices based on information included in the operation information ;
tracking, using the computing device, a location of the set of medical devices by obtaining the identifier information at the location, wherein the location of the set of medical devices includes a sterilization location, a medical operation location, and a decontamination location;
transmitting, via the computing device, the location of the set of medical devices to the medical instrument service;
updating the status of the set of medical devices in the repository based on the location;
in response to obtaining the identifier information from the barcode at the location, transmitting to the computing device, via the medical instrument service, information related to a sterilization operation when the location is the sterilization location, information related to a medical operation when the location is the medical operation location, and information related to a decontamination operation when the location is the decontamination location; and
providing, via a display, the information related to the sterilization operation when the location is the sterilization location, the information related to the medical operation when the location is the medical operation location, and the information related to the decontamination operation when the location is the decontamination location.

2. The method of claim 1, further comprising:
transmitting a query requesting identification of a first medical device within a facility associated with the computing device;
receiving identification of a second set of medical devices within the facility including the first medical device.

3. The method of claim 1, further comprising:
transmitting servicing information to the medical instrument service related to servicing the set of medical devices, wherein the servicing information is provided to the external service to trigger at least one operation for at least one medical device of the set of medical devices.

4. The method of claim 1, further comprising:
requesting status information from the medical instrument service pertaining to at least one status associated with sets of medical devices within a facility associated with the computing device; and
receiving the status information identifying performance of the at least one status within the facility.

5. The method of claim 4, wherein the status information includes performance metrics associated with at least one of at least one of sterilization operations, consignment operations, maintenance operations, or internal operations within the facility.

6. The method of claim 1, further comprising:
in response to obtaining the identifier information from the barcode, displaying information related to a current operation associated with the set of medical devices.

7. The method of claim 1, further comprising:
requesting an analysis of an operation type from the medical instrument service;
receiving previous information related to the operation type from the medical information, wherein the previous information includes identification of a configuration of each operation type, and wherein the configuration identifies at least set of medical devices.

8. The method of claim 7, wherein the analysis of the operation type includes a parameter associated with at least one of a patient, a medical condition, and the operation type.

9. The method of claim 7, wherein the previous information comprises information identifying at least one medical operator.

10. A non-transitory computer readable medium storing instructions that when executed by at least one processor, cause the at least one processor to perform operations for device management, the operations comprising:
transmitting a request for a set of medical devices from an external service using a medical instrument service;
receiving consignment information of the set of medical devices from the medical instrument service, wherein the external service provides information to the medical instrument service to trigger the consignment information;
when the set of medical devices is received, receiving identification information of the set of medical devices from the external service and providing the identification information and status information to a repository;
generating a label to couple to the set of medical devices, the label comprising medical device information and a barcode encoded with identifier information, wherein the medical device information comprises identification of medical events and least one person associated with the event, wherein the label is coupled to the set of medical devices, and wherein a status of the set of medical devices is changed in the medical instrument service in response to providing the label to the at least one processor;
obtaining information encoded into the barcode; in response to obtaining the identifier information from the barcode, transmitting, to the medical instrument service, operation information associated with a function of the set of medical devices, wherein the medical instrument service updates the set of medical devices based on information included in the operation information;
tracking a location of the set of medical devices by obtaining the identifier information at the location, wherein the location of the set of medical devices includes a sterilization location, a medical operation location, and a decontamination location;
transmitting the location of the set of medical devices to the medical instrument service;
updating the status of the set of medical devices in the repository based on the location;
in response to obtaining the identifier information from the barcode at the location, receiving, from the medical instrument service, information related to a sterilization operation when the location is the sterilization location, information related to a medical operation when the location is the medical operation location, and information related to a decontamination operation when the location is the decontamination location; and
providing, via a display, the information related to the sterilization operation when the location is the sterilization location, the information related to the medical operation when the location is the medical operation location, and the information related to the decontamination operation when the location is the decontamination location.

11. The non-transitory computer readable medium of claim 10, the operations further comprising:
transmitting a query requesting identification of a first medical device within a facility associated with the request for the set of medical devices; and
receiving identification of a second set of medical devices within the facility including the first medical device.

12. The non-transitory computer readable medium of claim 10, the operations further comprising transmitting service information to the medical instrument service related to servicing the set of medical devices, wherein the servicing information is provided to the external service to trigger at least one operation for at least one medical device of the set of medical devices.

13. The non-transitory computer readable medium of claim 10, the operations further comprising:
requesting status information from the medical instrument service pertaining to at least one status associated with sets of medical devices within a facility associated with the request for the set of medical devices; and
receiving the status information identifying performance of the at least one status within the facility.

14. The non-transitory computer readable medium of claim 13, wherein the status information includes performance metrics associated with at least one of at least one of sterilization operations, consignment operations, maintenance operations, or internal operations within the facility.

15. The non-transitory computer readable medium of claim 10, the operations further comprising in response to obtaining the identifier information from the barcode, displaying information related to a current operation associated with the set of medical devices.

16. The non-transitory computer readable medium of claim 10, the operations further comprising:
transmitting a request for an analysis of an operation type from the medical instrument service; and
receiving previous information related to the operation type from the medical information, wherein the previous information includes identification of a configuration of each operation type, and wherein the configuration identifies at least one set of medical devices.

17. The non-transitory computer readable medium of claim 16, wherein the analysis of the operation type includes a parameter associated with at least one of a patient, a medical condition, and the operation type.

18. The non-transitory computer readable medium of claim 16, wherein the previous information comprises at least one medical operator.

19. (canceled)

20. The non-transitory computer readable medium of claim 10, the operations further comprising repeating the operations at a new facility.

21. A method comprising:
requesting, using a computing device associated with a facility, a set of medical devices from an external service using a medical instrument service;
receiving consignment information of the set of medical devices from the medical instrument service, wherein the external service provides information to the medical instrument service to trigger the consignment information;
when the set of medical devices is received, obtaining identification information of the set of medical devices from the external service and providing the identification information and status information to a repository;
generating, using the computing device, a label for coupling to the label to the set of medical devices, the label comprising medical device information and a barcode encoded with identifier information, wherein the medical device information comprises identification of medical events and at least one person associated with the event, wherein the label is coupled to the set of medical devices, and wherein a status of the set of medical devices is changed in the medical instrument service in response to providing the label to the computing device;
obtaining, using the computing device, information encoded into the barcode;
in response to obtaining the identifier information from the barcode, transmitting, to the medical instrument service, operation information associated with a function of the set medical devices, wherein the medical instrument service updates the status of the set of medical devices based on information included in the operation information;
tracking, using the computing device, a location of the set of medical devices by obtaining the identifier information at the location, wherein the location of the set of medical devices includes a sterilization location, a medical operation location, and a decontamination location;
transmitting, via the computing device, the location of the set of medical devices to the medical instrument service;
updating the status of the set of medical devices in the repository based on the location;
in response to obtaining the identifier information from the barcode at the location, transmitting to the computing device, via the medical instrument service, information related to a sterilization operation when the location is the sterilization location, information related to a medical operation when the location is the medical operation location, and information related to a decontamination operation when the location is the decontamination location;
providing, via a display, the information related to the sterilization operation when the location is the sterilization location, the information related to the medical operation when the location is the medical operation location, and the information related to the decontamination operation when the location is the decontamination location;
transmitting, using the computing device, a request to the external service to retrieve the set of medical devices; and
repeating the method with the same set of medical devices at a new facility.
